(19) United States
(12) Patent Application Publication
(10) Pub. No.: US 2018/0296851 A1
(43) Pub. Date: Oct. 18, 2018

(54) VIRTUAL NATURE AT THERAPEUTIC BEST

(71) Applicants: Robert Bruce Carlin, Mission Viejo, CA (US); Susan Darlene Carlin, Mission Viejo, CA (US); Kevin Robert Carlin, San Diego, CA (US)

(72) Inventors: Robert Bruce Carlin, Mission Viejo, CA (US); Susan Darlene Carlin, Mission Viejo, CA (US); Kevin Robert Carlin, San Diego, CA (US)

(21) Appl. No.: 15/951,053

(22) Filed: Apr. 11, 2018

Related U.S. Application Data
(60) Provisional application No. 62/484,765, filed on Apr. 12, 2017.

(51) Int. Cl. A61N 5/06; A61M 16/00

(52) U.S. Cl. CPC A61N 5/06; A61N 2005/067; A61M 16/00

(57) ABSTRACT

This patent application describes a novel delivery system utilizing multiple natural therapeutic modalities featuring low-level laser light, clean enriched air with aromatic healing fragrances, and anti-inflammatory natural grounding currents from the surface of the earth, placed in omnipresent consumer devices, which conveniently bring these therapeutic modalities into close, frequent, and effective proximity to the consumer on a practical, economic, and safe platform that is independent of a doctor's office. This delivery system virtually brings nature at its therapeutic best into the comfort and convenience of the home and office where modern man virtually spends most of his existence.

### CROSS-REFERENCE TO RELATED APPLICATIONS

This application claims the benefit of U.S. Provisional Application No. 62/484,765, filed Apr. 12, 2017, which is hereby incorporated by reference, to the extent that it is not conflicting with the present application.

### BACKGROUND OF INVENTION

### 1. Field of the Invention

Nature provides an abundance of healing influences such as radiant light, aromatic fragrances, clean enriched air, and anti-inflammatory electrical currents from the earth's surface. However, modern man choosing to live in a darkened concrete castle has limited access to these healing influences. The novel interventions described in these applications will provide modern man trapped in his concrete castle with abundant and pleasant access to nature's healing influences in a practical and economical manner, utilizing devices and objects which are now omnipresent in every home, office, and exercise facility.

### BRIEF INVENTION SUMMARY

Described is a delivery system utilizing multiple natural therapeutic modalities featuring low-level laser light, clean enriched air with aromatic healing fragrances, and anti-inflammatory natural grounding currents from the surface of the earth, placed in omnipresent consumer devices, which conveniently bring these therapeutic modalities into close, frequent, and effective proximity to the consumer on a practical, economic, and safe platform that is independent of a doctor's office. These delivery systems virtually bring nature at its therapeutic best into the comfort and convenience of the home and office where modern man virtually spends most of his existence.

Certain wavelengths of light, such as 415 nm±40 nm, 633 nm±40 nm, and 880 nm±80 nm have demonstrated therapeutic gain in both human and animal studies with respect to significantly improved attention span, memory, mental processing speed, mood, treatment of traumatic brain injury, wound healing, photo-aging and wrinkles, excess pigmentation, acne, reduction of fat, repair of orthopedic injury and efficiency, and treating eye diseases such as macular degeneration. This therapeutic gain is predictable, given specificity of wavelength, adequate intensity, adequate proximity, and duration of treatment. With extended duration of treatment, the intensity can be reduced to levels that minimize the need for anatomical eye precautions and may even serve to be therapeutic in treating or preventing eye problems. This therapeutic gain, however, is limited by the lack of availability to the general population given a lack of delivery systems that allow for convenient, prolonged, and close approximation to a light source in a controlled manner, which minimizes risks and need for medical supervision by means of in-office delivery by a health provider.

This patent will describe novel methods of delivering therapeutic wavelengths cited above by integrating these wavelengths with the performance of devices constant before the public both professionally, and in-home at close ranges, allowing for efficient delivery of therapeutic wavelengths.

### DETAILED DESCRIPTION

Among the devices and objects appropriate for these novel methods are computer screens and their frames, keyboards and mice, television screens and frames, cellular phone screens and frames, picture frames with floral components, and exercise equipment with floral canopies placed in front and surrounding a television screen with its frame, demonstrating inspiring scenes from nature.

Within the context of computer screens and cellular phones, novel methods of delivery or novel combinations of supporting elements would involve the following:

Therapeutically potent LED lights in the frame around the computer or in after marketed device to be attached to the frame with controls to optimize intensity and tolerability and safeguards to prevent potential overdose in any 24-hour time period. The LED lights in the frame would also be unique, in that clinical significant therapeutic effects could be demonstrated and FDA device approval obtained. Eye protection or glare attenuation devices may be recommended for shorter, more intense exposure with warning messages issued per computer screen and other safeguards in place to guarantee that protective eye wear is in place. One enactment of these principles would involve placement of infrared LED lights in the keyboard area and in the frame of the computer screen focused on the face and nose of the user with internal programming to limit the flow energy and duration of energy to below the threshold of injury under worst-case conditions but allow for sufficient energy over 8 hours to exceed the therapeutic threshold of benefit. Among those benefits which would be scientifically and credibly demonstrated are increased vigilance and efficiency by 25% and better mood and memory by 30%. The sources of blue light and red light relevant to rejuvenation of skin and treatment of acne, could in this model be generated through the computer screen with program safeguards to verify that the intensity of energy or duration of energy never exceeded safe for maximum daily or alternatively red, white and blue light, could be concentrated over short duration of time with abundant visual cues and warnings to wear eye protection if at close range.

Software programs that create a frame on the computer screen, radiating therapeutic wavelengths with appropriate controls as cited above.

Software programs that will convert light waves showing a current content into light waves more closely approximating the therapeutic wavelengths cited above, except in scenes from nature where exact reproduction of color is to be prioritized.

Program sequences that run for five minutes every hour that fill the screen, emphasizing the therapeutic wavelengths organized into an entertaining or educational program, or an inspiring scene from nature that may also provide a refreshing needful break or rest time period from sustained eyestrain and intense problem solving. The therapeutic anti-inflammatory effects of red light at 633 nm emitted from the computer window can reasonably be combined with anti-inflammatory effect of a grounding pad under the forearm, such as marketed at www.grounding.com. Such grounding pads connect potentially and excessively inflamed human tissue with the negative electrons on the surface of the planet, which flow naturally and safely into the body that serve to neutralize injurious positively charged free radicals with safety comparable to walking barefoot on the beach. Computer screens produce electromagnetic frequency wavelengths that have been demonstrated to be injurious to sensitive individuals that can be ameliorated by grounding pads with benefits such as better mood and attention, and less arthritis.

Plants exist for more than human aesthetic and gratification, and are essential for human existence. Plants produce oxygen necessary for human respiration and life, but also to serve to clean the air effectively of pollutants, such as ultra-fine particulate matter from the diesel fuel that can cause respiratory disease, impairment of cognitive performance, and promote dementia as demonstrated in many recent human and animal studies. Plants also emit aromatic therapeutic compounds notable for raising mood and cognitive performance in ways both measurable and celebratory.

Within the context of picture frames containing floral elements that clean the air or emit therapeutic aromatic compounds, which can be found to elevate mood and mental efficiency. LED lights could be placed within that will serve to grow the plants, as well as provide therapeutic wavelengths described above, acting in synergy with the aromatic compounds emitted from the therapeutic plants. Grounding pads or attachments could be attached to the frame, which will serve to enhance growth and productivity of the functional plants attached to the frame and/or placed upon a desk, chair, or floor nearby to directly contact the individual person approximate to the picture frame. Such picture frames would typically be placed on the wall adjacent to a desk as in an office cubicle, so as to create proximity to the person utilizing the desk. and benefiting from exposure to the low-level laser light radiation from the LED lights. This multiple platform picture frame can additionally be applied not only to a conventional stable picture, but also to a television screen program to show a variety of works of art emphasizing blue, red, and infrared therapeutic wavelengths.

Within the context of floral canopies surrounding a television screen place in front of exercise equipment, such a treadmill or stair-stepper, LED lights could be placed here to serve to grow the plants in addition to provide therapeutic wavelengths described above in synergy with aromatic compounds emitted from the therapeutic plants, and serve to provide virtual nature at its therapeutic best. The exercise equipment could be enhanced therapeutically by combining exercise with grounding pads attached to the upper arm or waist area, which will serve to diminish excessive inflammation created through exercise, in combination to treat arthritic joints. Grounding wires can also be attached to the functional floral plants in the canopy in front of the treadmill to stimulate growth of productivity of the functional plants.

The uniqueness of this patent, in part, can be found in the novel or unique combination of several natural clinically potent therapeutic modalities placed in omnipresent consumer devices, which conveniently bring these therapeutic modalities into close, frequent, and effective proximity to the consumer in a practical, economical, and safe platform that is independent of a doctor's office. ide therapeutic wavelengths described above in synergy with aromatic compounds emitted from the therapeutic plants, and serve to provide virtual nature at its therapeutic best. The exercise equipment could be enhanced therapeutically by combining exercise with grounding pads attached to the upper arm or waist area, which will serve to diminish excessive inflammation created through exercise, in combination to treat arthritic joints. Grounding wires can also be attached to the functional floral plants in the canopy in front of the treadmill to stimulate growth of productivity of the functional plants.

The uniqueness of this patent, in part, can be found in the novel or unique combination of several natural clinically potent therapeutic modalities placed in omnipresent consumer devices, which conveniently bring these therapeutic modalities into close, frequent, and effective proximity to the consumer in a practical, economical, and safe platform that is independent of a doctor's office.

What is claimed is:

1. A delivery system utilizing multiple natural therapeutic modalities comprising laser light, clean enriched air with aromatic healing fragrances, and anti-inflammatory natural grounding currents from the surface of the earth, the delivery system being placed in a consumer device positioned in a proximity of a consumer.
